(19) United States
(12) Patent Application Publication
(10) Pub. No.: US 2008/0003286 A1
(43) Pub. Date: Jan. 3, 2008

(54) SUSTAINED DELIVERY ALFUZOSIN COMPOSITIONS

(76) Inventors: Sathya Narayana Vemula, Hyderabad (IN); Uma Devi Palaparthi, Hyderabad (IN); Venkat Arvind Vakati, Hyderabad (IN); Sarveswara Rao Srirama Mandavilli, Hyderabad (IN); Nookaraju Venkata Sreedharala, Hyderabad (IN); Srinivas Arutla, Hyderabad (IN); Rajesh Gunde Rao Kulkarni, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/427,400

(22) Filed: Jun. 29, 2006

(51) Int. Cl. A61K 9/24; A61K 31/517

(52) U.S. Cl. 424/472; 514/252.17

(57) ABSTRACT

Controlled release pharmaceutical compositions containing alfuzosin.

### INTRODUCTION TO THE INVENTION

The present invention relates to the controlled release pharmaceutical compositions of alfuzosin or pharmaceutically acceptable salts, solvates, enantiomers or mixtures thereof and processes for preparing the same.

Alfuzosin, chemically known as (+)-N-[3-[(4-Amino-6,7-dimethoxy-2-quinazolinyl) methylamino]propyl] tetrahydro-2-furancarboxamide monohydrochloride, is useful in the treatment of benign hypertrophy of the prostate, and is commercially available as 2.5 mg immediate release tablets; 5 mg and 10 mg controlled release tablets. Controlled release formulations are available commercially under the trademarks XATRAL-XL in Europe and UROXATRAL in the USA.

Alfuzosin has a short half-life and demonstrates preferential absorption in the upper gastrointestinal tract, with more intense absorption at the duodenum-jejunum level.

Controlled release drug delivery systems are among the pharmaceutical drug delivery systems that are useful in delivering those active pharmaceutical ingredients that have a narrow therapeutic range, short biological half-life, and/or high toxicity. The said systems allow the dosage delivery by reducing the frequency of administration, and provide the desired therapeutic effect throughout the day. This is especially true of highly water-soluble compounds.

U.S. Pat. No. 6,149,940 discloses a controlled release composition of alfuzosin in the form of a triple layer tablet comprising a hydrophilic active matrix core containing alfuzosin and two functional layers, one an erodible layer and the other a swellable layer. International Application Publication No. WO 2004/037228 discloses a controlled release composition of alfuzosin comprising a single functional layer containing alfuzosin and optionally one or more non-functional layers adjacent to the single functional layer.

U.S. Pat. No. 4,642,233 discloses a controlled delivery device comprising a swellable hydrogel reservoir that expands in a fluid environment to a very high degree (2- to 50-fold), and containing a plurality of tiny pills, which can release a drug by a dual release principle. The tiny pills comprise a drug plus a release rate controlling membrane.

A pharmaceutical composition for providing a controlled release of alfuzosin, through the use of a dual-control release principle to deliver alfuzosin in a more consistent and reliable manner by residing in a proximal part of the gastrointestinal tract would be a significant improvement in the formulation of alfuzosin and in its use in the therapy of benign prostatic hypertrophy.

### SUMMARY OF THE INVENTION

The present invention relates to the pharmaceutical compositions of alfuzosin or pharmaceutically acceptable salts, solvates, enantiomers or mixtures thereof based on dual retarding controlled release principle.

Another aspect of the invention provides a pharmaceutical composition of alfuzosin or pharmaceutically acceptable salts, solvates, enantiomers or mixtures thereof with improved drug release characteristics.

In an aspect, a pharmaceutical composition of the invention comprises multiple reservoirs comprising a coating comprising alfuzosin applied onto a pharmaceutically inert particle, and having an outer hydrophilic or hydrophobic coating, dispersed in an external matrix composition comprising a hydrophilic polymer.

In a further aspect, a pharmaceutical composition of the invention comprises:

a) a solid pharmaceutically inert particle having an intermediate coating comprising alfuzosin and a polymer, and having an outer coating comprising a hydrophobic polymer; dispersed in

b) an external matrix composition comprising hydroxypropyl methylcellulose and a polysaccharide.

### DETAILED DESCRIPTION OF THE INVENTION

The present invention provides a pharmaceutical formulation for alfuzosin or pharmaceutically acceptable salts, solvates, enantiomers or mixtures thereof with an improved and predictable controlled release dissolution profile.

The said formulation comprises multiple reservoirs embedded in a matrix composition, wherein both reservoir and matrix compositions control the release of alfuzosin. Such a formulation that uses a dual retard technique leads to significantly improved pharmaceutical compositions overcoming shortcomings such as risk of dose dumping and burst release.

The present invention provides alfuzosin in multiple reservoir systems of defined size that are coated with a coating material to a defined coating built up, and the multiple reservoirs are embedded in a matrix composition with or without use of other pharmaceutically accepted excipients.

“Reservoir systems” as used herein are defined as any of:
a) Particles or beads coated with alfuzosin and subsequently coated with a rate controlling substance.
b) Particles or beads coated with a combination of alfuzosin and a rate controlling substance.
c) Alfuzosin and a rate controlling substance, with or without other pharmaceutically acceptable excipients, being blended, granulated, spheronised or extruded to form a drug reservoir.

Multiple reservoir systems may be produced by any technique known in the art including but not limited to extrusion, spheronisation, fluid bed coating, conventional coating, solution spraying, drenching, powder spraying, including top, bottom or tangential coating techniques and the like.

The reservoir systems herein can have a size in the range of 10 μm to 1 mm, or 150 μm to 400 μm.

Reservoir systems of the present invention are prepared from pharmaceutically inert particulate components such as but not limited to microcrystalline cellulose (MCC), dicalcium phosphate, tricalcium phosphate, sugar crystals, sugar globules, mannitol, microcrystalline cellulose spheres (e.g. CELPHERE™, a product of Asahi Kasei Chemicals Corporation, Tokyo, Japan), silicon dioxide particles and the like.

Alfuzosin is applied as a coating on the inert particles, such as in a liquid composition comprising the drug, and a water-soluble or water-dispersible polymer. The alfuzosin coating can be an intermediate coating or the only coating on the particles.

The pharmaceutical compositions of the present invention may contain one or more hydrophobic outer coating materials on the reservoir particles used for retarding the release of drug from reservoirs, such as but not limited to waxes such as carnauba wax and white wax, fatty alcohols, fatty acid esters, glycerol monooleate, acetylated monoglycerides, glyceryl behanate, glyceryl monostearate, hydrogenated vegetable oils, paraffin, cellulose polymers such as ethylcellulose, and the like.

The pharmaceutical compositions of the present invention may contain one or more hydrophilic outer coating materials on the reservoir particles useful for retarding release of the drug from reservoirs such as but not limited to various grades of poly(N-vinylpyrrolidone), polyacrylamide, polyethylene oxide, acrylic acid, acrylic acid derivatives, acrylic acid esters, polyacrylic acid, cellulose polymers such as carboxymethyl cellulose, hydroxypropyl cellulose, hydroxylethylcellulose, hydroxypropyl methylcellulose, methyl cellulose, polyvinyl alcohol, polyvinyl pyrrolidone, polyethylene glycol and the like.

The amount of hydrophilic or hydrophobic outer coating build up in reservoir systems can generally range from 1% to 50%, or from 5% to 25%, or from 10% to 20%, by weight of the reservoir composition.

Matrix compositions used in the present invention are intended to contribute to the release profile of alfuzosin from the composition and also help in prolonging the residence time in the gastrointestinal tract.

External matrix compositions may be prepared by simple dry mixing of a hydrophilic polymer and other pharmaceutically acceptable excipients, dry granulation or wet granulation using aqueous, hydro alcoholic or solvent based granulation using conventional granulation techniques such as rapid mixer granulation, planetary mixing, mass mixing, fluid bed granulation and the like.

Hydrophilic polymers or combinations thereof used in various ratios are exemplified by but are not limited to cellulose polymers such as various grades of methyl cellulose, hydroxyethyl cellulose, hydroxypropyl cellulose, hydroxypropyl methylcellulose (HPMC); homo- or co-polymers of N-vinyl pyrrolidones such as polyvinyl pyrrolidone; polyacrylic acid and its derivatives; copolymers of polyethylene glycols, polyorthoesters, polyurethanes and the like.

In the external matrix composition, polysaccharide materials such as but not limited to lactose, mannitol, sorbitol, maltitol, microcrystalline cellulose and powdered cellulose can be used to modulate the rate of release of alfuzosin, and the amount of such material with respect to the matrix composition can range from 5% to 60%, or from 10% to 50% by weight.

The multiple reservoirs and external matrix composition along with other pharmaceutically acceptable excipients can be formulated into a suitable solid oral dosage form such as tablets and the like by procedures known to a person skilled in the art of preparation of pharmaceutical formulations. Such compositions can include other excipients as are required for the preparation of the compositions including but not limited to diluents, granulating agents, solvents, lubricants, wetting agents, disintegrating agents and the like, and any combinations thereof.

The pharmaceutical compositions of the present invention may contain one or more diluents added to increase mass and, hence, provide easier handling for the patient and caregiver. Common diluents are microcrystalline cellulose, microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, potassium chloride, powdered cellulose, sodium chloride, sorbitol, talc and the like.

Binders also can be included in the pharmaceutical compositions of the present invention. Some typical binders are acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methylcellulose (HPMC) (e.g. Methocel®), liquid glucose, magnesium aluminum silicate, maltodextrin, methylcellulose, polymethacrylates, polyvinylpyrrolidone (PVP or povidone, e.g. Kollidon®, Plasdone®), pregelatinized starch, sodium alginate, starch and the like.

Pharmaceutical compositions for tableting and film formation may include without limitation, any one or more of pharmaceutically acceptable glidants, lubricants, flavoring agents, plasticizers, opacifiers, colorants and other commonly used excipients.

In an embodiment, the present invention provides a unit dose of alfuzosin of about 0.1 to about 20 milligrams per dosage form.

The following examples will further describe certain specific aspects and embodiments of the invention in greater detail and are not intended to limit the scope of the invention.

### EXAMPLE 1

### Preparation of a Controlled Release Pharmaceutical Composition of Alfuzosin Hydrochloride.

### A) Composition of Drug Reservoirs:

| Ingredients | mg/Tablet |
|---|---|
| Alfuzosin hydrochloride | 10 |
| HPMC 3 Cps | 2 |
| Celphere 203* | 76 |
| Aquacoat^{#} | 14 |
| Triethyl citrate | 2 |
| Water | 100 |

*CELPHERE 203, from Asahi Kasei, are microcrystalline cellulose spheres having sizes between 150 μm and 300 μm.
#AQUACOAT ™ from FMC Corporation, Philadelphia, Pennsylvania U.S.A., is an aqueous dispersion of ethylcellulose polymer, containing 24.5–29.5 wt. percent ethylcellulose.

1. Alfuzosin Hydrochloride was dispersed in water and stirred until it was dissolved.
2. HPMC 3 Cps was separately dissolved in water.
3. Both the solutions were mixed and stirred for 15 min.
4. Triethyl citrate was added to Aquacoat and mixed.
5. Water was added to the dispersion of step 4 and mixed.

### B) Manufacturing Process of Drug Reservoirs:

6. The solution of step 3 was sprayed onto the Celpheres.
7. The coated Celpheres of step 6 were further coated with mixture of step 5 in a fluid bed coater using the following coating parameters: ???

### C) Composition of Matrix System:

| Ingredients | mg/Tablet |
|---|---|
| HPMC K 100M | 35 |
| HPMC K 4M | 131 |
| Avicel PH101* | 91 |
| Pharmatose DCL15** | 40 |
| PVP K 90D | 40 |
| Isopropyl alcohol (IPA) | 180 |
| Magnesium stearate | 9 |
| Drug reservoirs | 104 |

*Avicel PH101, from FMC Corporation, Philadelphia, Pennsylvania U.S.A. is microcrystalline cellulose where ASTM mesh #60 sieve retains not more than 1% and ASTM mesh #200 sieve retains less than or equal to 30%.
**Pharmatose DCL 15, from DMV International, Netherlands is free flowing, directly compressible lactose.

### D) Manufacturing Process of Final Composition:

8. HPMC-K100M, HPMC-K4M, MCC and lactose were sieved through an ASTM mesh #40 sieve.
9. PVP K 90D was dissolved in IPA with continuous stirring and mixed with mixture of step 8 and subsequently granulated.
10. The granules were dried at 60° C. for 30 minutes or till the loss on drying (LOD) was below 3% when tested at 105° C. to constant weight using an infrared moisture balance.
11. The dried granules were sieved through an ASTM mesh #30 sieve.
12. The coated pellets of step 7 were also passed through an ASTM mesh #30 sieve.
13. The granules of step 11 were blended with pellets of step 12 for 5 minutes along with magnesium stearate previously sifted through an ASTM mesh #80 sieve.
14. The blend of step 13 was compressed using 10 mm round shaped flat faced beveled edge punches on a rotary tablet compression machine, providng the following tablet parameters:

| Average weight of tablet | 450 mg ± 5% |
|---|---|
| Hardness (KP) | 4–10 |
| Thickness (mm) | 4–5 |

### EXAMPLE 2

### In-Vitro Dissolution Study Using the Composition of Example 1.

The in-vitro release of alfuzosin hydrochloride from the composition of Example 1 was studied at pH 6.8 using Apparatus 2 of Procedure 711 (Dissolution) of The United States Pharmacopoeia 24, United States Pharmacopoeial Convention, Rockville, Md. U.S.A. 2000.

| Time (hours) | Drug Release (%) |
|---|---|
| 1 | 11.3 |
| 2 | 18 |
| 4 | 29.5 |
| 6 | 39 |
| 8 | 47.6 |
| 10 | 56 |
| 12 | 63 |
| 16 | 76.8 |
| 20 | 88 |
| 24 | 98 |

### EXAMPLE 3

### Preparation of a Controlled Release Pharmaceutical Composition of Alfuzosin Hydrochloride Comprising a Higher Concentration of HPMC.

a) Composition of the Drug Reservoirs:

| Ingredient | mg/Tablet |
|---|---|
| Alfuzosin hydrochloride | 10 |
| Celphere CP 203 | 70.4 |
| HPMC (3 cps) | 3.6 |
| Water | 100 |

1. Alfuzosin hydrochloride was dissolved in water with stirring.
2. HPMC 3 cps was dispersed in water with stirring.
3. Solution of step 1 was added to the dispersion of step 2 and stirred for 15 minutes.
4. The mixture was filtered through a mesh #200 nylon cloth.
5. The solution of step 4 was sprayed onto the Celpheres

b) Polymer Coating:

| Ingredient | mg/Tablet |
|---|---|
| Drug loaded pellets | 84 |
| Aquacoat | 13.44 |
| Triethyl citrate | 1.92 |
| Water | 60 |

6. Triethyl citrate was added to Aquacoat and stirred for 15 minutes.
7. Water was added to the above dispersion.
8. Stirring was continued for 15 minutes and the mixture was filtered through a mesh #200 nylon cloth.
9. The drug coated Celpheres were further coated with the mixture of step 8.

c) Composition of the Matrix System:

| Ingredient | mg/Tablet |
|---|---|
| HPMC K 100 M | 35 |
| HPMC K 4 M | 141 |
| Microcrystalline cellulose | 95 |
| 114 | |
| Lactose DT* | 30 |
| PVP K 90D | 40 |
| Isopropyl alcohol | 180 |

*Direct Tableting

10. HPMC K 100M, HPMC K4M, MCC 114 and Lactose DT were sifted through an ASTM mesh #40 sieve.
11. PVP K 90D was dissolved in IPA while stirring.
12. The granules were dried in a fluid bed drier till the loss on drying (LOD) at 105° C. was less than 3%.
13. The dried granules were sifted through an ASTM mesh #30 sieve.

d) Preparation of the Final Dosage Form:

| Ingredient | mg/Tablet |
|---|---|
| Polymer coated pellets of | 100 |
| step 9 | |
| Granules of step 13 | 341 |
| Magnesium stearate | 9 |

14. Polymer coated pellets and the sifted granules of step 5 were blended in a blender for 20 minutes.
15. Magnesium stearate was passed through an ASTM mesh # 80 sieve and added to the blend of step 12.
16. Blending was continued for 5 minutes.
15. The blend was compressed using 10 mm round punches on a rotary tablet compression machine, giving the following tablet parameters:

| Average weight of tablet | 450 mg ± 5% |
|---|---|
| Hardness (KP) | 4–12 |
| Thickness (mm) | 4–5 |

### EXAMPLE 4

### In-Vitro Dissolution Study Using the Composition of Example 3.

In-vitro dissolution of the composition of Example 3 was compared with that of commercial UROXATRAL® 10 mg capsules (Batch No. TH35). The study was performed using a USP type II (paddle) dissolution apparatus and 900 ml of pH 6.8 phosphate buffer at 37° C.±0.5° C. as the dissolution medium.

| | |
|---|---|
| Time | Drug Release (%) |

| (hours) | Composition of Example 3 | UROXATRAL ® 10 mg |
|---|---|---|
| 1 | 15 | 13 |
| 4 | 29 | 30 |
| 8 | 43 | 46 |
| 16 | 68 | 68 |
| 24 | 92 | 85 |

### EXAMPLE 5

### Bioequivalence Study of the Composition of Example 3.

A randomized, open-label, balanced, two-treatment, two-period, two-sequence, single dose, crossover bioequivalence study of compositions of Example 3 (“Test”) with UROXATRAL® (“Reference”) 10 mg alfuzosin extended release tablets from Sanofi-Synthelabo, Inc. in twenty-four healthy, adult male, human subjects under fasting conditions. The washout period between treatments was seven days.

| Pharmacokinetic | | |
|---|---|---|
| Parameter | Test | Reference |
| Tₘₐₓ (hours) | 7.54 | 8.94 |
| Cₘₐₓ (ng/ml) | 15.33 | 18.37 |
| AUC_{0–t} (ng · h/ml) | 170.64 | 195.37 |
| AUC_{0–∞} (ng · h/ml) | 181.32 | 203.66 |

### EXAMPLE 6

### Preparation of a Controlled Release Pharmaceutical Composition of Alfuzosin Hydrochloride Coated with Surelease™.

| | |
|---|---|
| Ingredient | mg/Tablet |

Composition of drug reservoir

| | |
|---|---|
| Alfuzosin hydrochloride | 10 |
| Celphere CP 203 | 70.4 |
| HPMC (3 cps) | 3.6 |
| Water | 100 |

Polymer coating

| | |
|---|---|
| Drug loaded pellets | 84 |
| Surelease ™* | 15.36 |
| Water | 60 |

Composition of matrix system

| | |
|---|---|
| HPMC K 100 M | 35 |
| HPMC K 4 M | 141 |
| Microcrystalline cellulose | 95 |
| 114 | |
| Lactose DT | 30 |
| PVP K 90D | 40 |
| Isopropyl alcohol | 180 |

Preparation of final dosage form

| | |
|---|---|
| Polymer coated pellets of | 100 |
| step 9 | |
| Granules of step 13 | 341 |
| Magnesium stearate | 9 |

*Surelease is a commercially available 25% w/w ethylcellulose dispersion in ammoniated water, marketed by Colorcon Ltd. The other components of the dispersion are oleic acid and dibutyl sebacate.

The composition is prepared in a similar manner to that of Example 3 except that Surelease® is used for coating of drug loaded Celpheres of step 5. Further, steps 10 through 16 are followed as in Example 3.

### EXAMPLE 7

### Preparation of Controlled Release Pharmaceutical Composition of Alfuzosin Hydrochloride Comprising Ethyl Cellulose

| | |
|---|---|
| Ingredient | mg/Tablet |

Composition of drug reservoir

| | |
|---|---|
| Alfuzosin hydrochloride | 10 |
| Celphere CP 203 | 70.4 |
| HPMC (3 cps) | 3.6 |
| Water | 100 |

Polymer coating

| | |
|---|---|
| Drug loaded pellets | 84 |
| Ethyl cellulose | 9.36 |
| HPMC 3 cps | 4.8 |
| Triethyl citrate (TEC) | 1.74 |
| Isopropyl alcohol | 160 |
| Methylene chloride | 52.36 |

Composition of matrix system

| | |
|---|---|
| HPMC K 100 M | 35 |
| HPMC K 4 M | 141 |
| Microcrystalline cellulose | 95 |
| 114 | |
| Lactose DT | 30 |
| PVP K 90D | 40 |
| Isopropyl alcohol | 180 |

Preparation of final dosage form

| | |
|---|---|
| Polymer coated pellets of | 100 |
| step 9 | |
| Granules of step 13 | 341 |
| Magnesium stearate | 9 |

The compositions are prepared in a similar manner to that of Example 3 except that the drug loaded Celpheres of step 5 are coated with coating mixture prepared by dissolving ethylcellulose and HPMC in a solvent mixture of isopropyl alcohol and methylene chloride and further addition of TEC to this mixture. Further, steps 10 through 16 are followed as in Example 3.

We claim:

1. A pharmaceutical composition comprising:
multiple reservoirs comprising a coating comprising alfuzosin applied onto a pharmaceutically inert particle, and having an outer hydrophilic or hydrophobic coating, dispersed in
an external matrix composition comprising a hydrophilic polymer.

2. The pharmaceutical composition of claim 1, wherein a pharmaceutically inert particle comprises microcrystalline cellulose.

3. The pharmaceutical composition of claim 1, wherein reservoirs have sizes from about 10 μm to about 1 mm.

4. The pharmaceutical composition of claim 1, wherein an outer coating comprises ethylcellulose.

5. The pharmaceutical composition of claim 1, wherein an outer coating comprises about 1 to about 50 percent by weight of a reservoir.

6. The pharmaceutical composition of claim 1, wherein an external matrix composition comprises hydroxypropyl methylcellulose.

7. The pharmaceutical composition of claim 1, wherein an external matrix composition further comprises at least one polysaccharide.

8. The pharmaceutical composition of claim 1, wherein an external matrix composition further comprises a cellulose.

9. The pharmaceutical composition of claim 1, wherein an external matrix composition further comprises at least one of a diluent and a binder.

10. The pharmaceutical composition of claim 1, which is in the form of a tablet.

11. The pharmaceutical composition of claim 1, wherein a pharmaceutically inert particle comprises microcrystalline cellulose, an outer coating comprises ethylcellulose, and an external matrix composition comprises hydroxypropyl methylcellulose.

12. The pharmaceutical composition of claim 11, wherein an external matrix composition further comprises a polysaccharide.

13. The pharmaceutical composition of claim 11, wherein an external matrix composition further comprises a cellulose.

14. A pharmaceutical composition, comprising:
a) a solid pharmaceutically inert particle having an intermediate coating comprising alfuzosin and a polymer, and having an outer coating comprising a hydrophobic polymer; dispersed in
b) an external matrix composition comprising hydroxypropyl methylcellulose and a polysaccharide.

15. The pharmaceutical composition of claim 14, wherein an intermediate coating comprises alfuzosin and hydroxypropyl methylcellulose.

16. The pharmaceutical composition of claim 14, wherein an outer coating comprises ethylcellulose.

17. The pharmaceutical composition of claim 14, wherein coated pharmaceutically inert particles of a) have sizes from about 10 μm to about 1 mm.

18. The pharmaceutical composition of claim 14, wherein a polysaccharide comprises microcrystalline cellulose.

19. The pharmaceutical composition of claim 14, wherein an external matrix composition further comprises a diluent, granulating agent, lubricant, wetting agent, disintegrating agent, or any combination of two or more thereof.

20. The pharmaceutical composition of claim 14, which is in the form of a tablet.
